(19) United States
(12) Patent Application Publication
(10) Pub. No.: US 2025/0074877 A1
(43) Pub. Date: Mar. 6, 2025

(54) METHODS AND COMPOSITIONS FOR TREATMENT OF PREECLAMPSIA AND CANCER

(71) Applicant: Akkadian Therapeutics, Inc., Stoneham, MA (US)

(72) Inventors: Prakash G. Jagtap, North Andover, MA (US); Kanneganti Murthy, Stoneham, MA (US); Garry J. Southan, Swampscott, MA (US)

(21) Appl. No.: 18/818,334

(22) Filed: Aug. 28, 2024

Related U.S. Application Data
(60) Provisional application No. 63/579,119, filed on Aug. 28, 2023.

(51) Int. Cl. C07D 211/94; A61K 31/45; A61K 31/454; A61K 45/06; A61P 35/00

(52) U.S. Cl. CPC C07D 211/94; A61K 31/45; A61K 31/454; A61K 45/06; A61P 35/00

(57) ABSTRACT

Provided herein, in various embodiments, are compounds of general formula I:

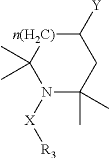

for use in the treatment of preeclampsia and preeclampsia associated conditions eclampsia, haemorrhagic stroke, haemolysis, elevated liver enzymes and low platelet count (HELLP) syndrome, placental abruption, renal failure and pulmonary oedema. Also described in embodiments herein are compounds of general formula II

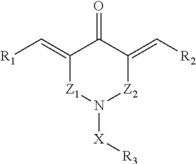

or general formula III

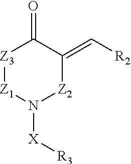

for use in the prevention of cancer.

### RELATED APPLICATIONS

This application claims the benefit of U.S. Provisional Application No. 63/579,119, filed on Aug. 28, 2023. The entire teachings of the above application(s) are incorporated herein by reference.

### TECHNICAL FIELD

The present disclosure provides methods and compositions for the treatment of preeclampsia, as well as therapeutic and prophylactic methods of use thereof such as in the treatment or prevention of cancer.

### BACKGROUND ART

Preeclampsia (PE) is a pregnancy complication characterized by high blood pressure and signs of damage to other organ systems, most often the liver and kidneys, dysfunction, proteinuria, and in severe cases, eclampsia and premature birth. Preeclampsia usually begins after 20 weeks of pregnancy in women whose blood pressure had been normal. While the etiology and pathogenesis of PE are elusive, placental ischemia/hypoxia due to impaired placental development and subsequent release of anti-angiogenic factors (sFLT1 and sEng) are key components. There is growing evidence that placental ischemia, due to impaired spiral artery remodeling and abnormal secretion of anti-angiogenic factors, impairs trophoblast mitochondrial function and energy production, leading to the release of reactive oxygen species (ROS). ROS has been shown to stabilize hypoxia inducible factor (HIF1A), which in turn can induce transcription of anti-angiogenic factors, soluble fms-like tyrosine kinase 1 (sFLT1) and soluble endoglin in trophoblasts. The ROS and reactive nitrogen species (RNS) originate from mitochondria in preeclamptic placentas, causing impaired mitochondrial function in both humans and in animal models of PE. Therefore, nitroxide and nitric oxide (NO) donor compounds and their prodrugs may counteract the oxidant stress both in the kidney and placenta in our model, reducing HIF1A production, which in turn may prevent the anti-angiogenic response—sFlt1 production—and the endothelial dysfunction, all major factors in PE pathogenesis [Ref 1]. These compounds and their prodrugs will target both the imbalance in oxygen-centered and nitrogen-centered free radicals, and the maternal hypertension, thereby acting on two components underlying the pathophysiology of preeclampsia.

Many chemotherapeutic agents act by producing free radicals, which may increase oxidative stress in normal cells. To minimize this toxicity, there is a need to use detoxicants, such as antioxidants, which can differentiate between healthy and cancerous cells and selectively protect the healthy cells by scavenging free radicals. Nitroxides, a class of small-molecular-weight heterocyclic molecules containing “NO·,” nitroxide and hydroxylamines, the one electron-reduced form of nitroxides characterized by “NOH,” have shown preferentially scavenge oxygen radicals in cells that have normal oxygenation or redox status. The reduced form of nitroxide called hydroxylamine, which exists in equilibrium with the nitroxide form in well oxygenated tissues. Most tumors are hypoxic in nature and their cellular environment is more reducing and carry thiol reach environment when compared with healthy cells [Ref 2]. This differential aspect between normal and cancerous cells has led us to the design of compounds, which would have both anticancer and antioxidant properties.

### SUMMARY

In one aspect, the present disclosure provides compounds of the general formulae I, II and III:

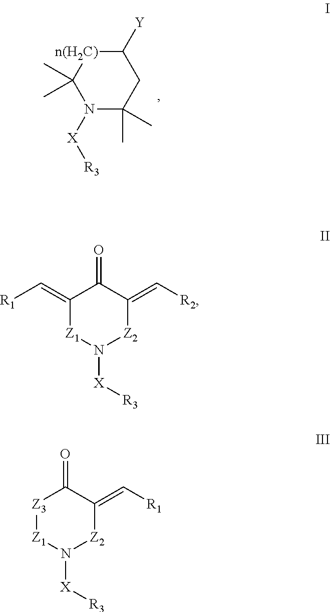

or enantiomers, diastereomers, racemates, or a pharmaceutically acceptable salt thereof,
wherein:
X is selected from O·, or —OH or H wherein R3 is absent or X is selected from CO, OCO, SO2, OSO2, CONH—, OCONH, —CO-aryl or heterocyclic ring, OCO-aryl or heterocyclic, CO(CH2)n, CO(CH2)nCO, OCO(CH2)nCO, OCO(CH2)n—;
R3 is substituted amines such as methylamine, dimethyl, trimethyl, ethylamine, diethyl, propylamine, isopropylamine, pyrrolidine, piperazine, morpholine, and nitroxide containing amines such as 3-amino-proxyl, 3-aminomethyleneproxyl, 4-amino-TEMPO, and amino acids such as glycine, valine, arginine, proline and cystine linked via amides and phosphate salts or alkyl esters carrying substituted amines, amino acids, amides and phosphate salts;
Y is selected from H, ═O, —OH, CH2OH, —CO2H, —ONO₂, —CH₂ONO₂, —CH₂CH₂ONO₂, CH═CH—CH₂ONO₂, COOCH₂CH₂ONO₂ and —COOalkyl where said alkyl may be optionally substituted, for example, with —ONO₂;

Z1, Z2, and Z3 are independently selected from CH2, —CF2, CMe2, CO, CHCONHR3, CH—COCH═CHR1, CH—COCH═CHR2, C═CH—R1 or C═CH—R2 or Z1 and Z2 taken together form a 5- or 6 membered carbocyclic or heterocyclic ring or

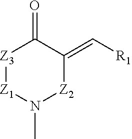

ring is selected from the following:

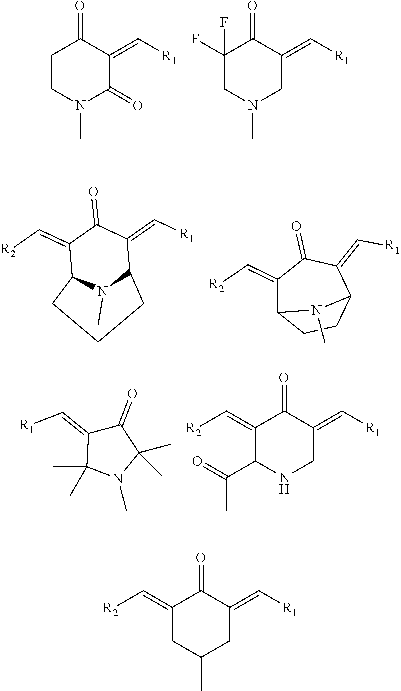

n is 0 to 4.

R₁ and R₂ are independently an alkyl, 5 or 6 membered aromatic heterocyclic or carbocyclic rings optionally substituted with OH, OMe, F, CF3, CN, NO₂, CO₂Et or SO₂Me groups.

In an embodiment, R₁ and R₂ are either on same side of the double bond, or on opposite sides of the double bond, with E/E, E/Z or Z/Z configuration and any alkyl, 5 or 6 membered aromatic heterocyclic and carbocyclic rings substituted with H, OH, OMe, F, CF3, CN, NO₂, CO₂Et or SO₂Me groups.

In other embodiments, R3 is substituted H or C1 to C10 alkyl or aryl or heterocyclic rings.

Compounds of the disclosure can be used in the treatment of preeclampsia, cardiovascular diseases and prevention of cancer. In an embodiment, a method for treating preeclampsia or preeclampsia associated conditions comprising eclampsia, haemorrhagic stroke, haemolysis, elevated liver enzymes and lowe platelet count (HELLP) syndrome, placental abruption, renal failure or pulmonary oedema, the method comprising administering a compound of the general formula I or enantiomers, diasteromers, racemates or a pharmaceutically acceptable salt thereof to a patient in need.

In another aspect, the present disclosure provides a pharmaceutical composition comprising a compound of the general formula I and II as defined above, or an enantiomer, diastereomer, racemate, or a pharmaceutically acceptable salt thereof, and a pharmaceutically acceptable carrier, for treatment of preeclampsia, and preeclampsia associated conditions eclampsia, haemorrhagic stroke, haemolysis, elevated liver enzymes and low platelet count (HELLP) syndrome, placental abruption, renal failure and pulmonary oedema, and prevention of cancer.

In yet another aspect, the present disclosure relates to use of a compound of the general formula I and II as defined above, or an enantiomer, diastereomer, racemate, or a pharmaceutically acceptable salt thereof, for the preparation of a pharmaceutical composition for treatment of preeclampsia, pulmonary hypertension and pneumonia.

In a further aspect, the present disclosure relates to a method for treatment of preeclampsia and cancer in an individual in need thereof, comprising administering to said individual an effective amount (about 1 mg to about 750 mg/kg) of a compound of the general formula I and II as defined above, or an enantiomer, diastereomer, racemate, or a pharmaceutically acceptable salt thereof in combination with other anti-cancer agents.

### DETAILED DESCRIPTION

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless the context requires otherwise, throughout the specification and claims that follow, the word “comprise” and synonyms and variants thereof such as “have” and “include”, as well as variations thereof, such as “comprises” and “comprising”, are to be construed in an open, inclusive sense, e.g., “including, but not limited to.” The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element or step not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the disclosure, claims, result or embodiment, “about” means within one standard deviation per the practice in the art, or can mean a range of ±20%, ±10%, ±5%, ±4, ±3, ±2 or ±1% of a given value. It is to be understood that the term “about” can precede any particular value specified herein, except for particular values used in the Examples.

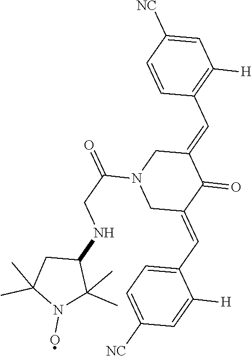

indicates an enantiomer, that is, either of the two representations below:

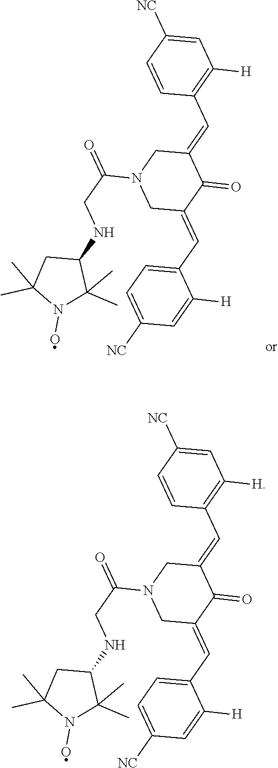

### Likewise,

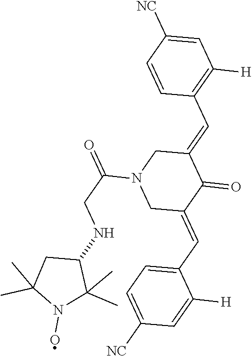

is the other enantiomer of and is either of

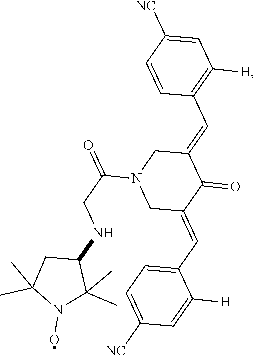

the two representations below:

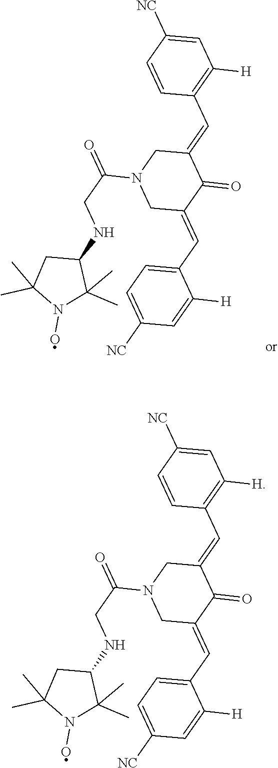

The representation:

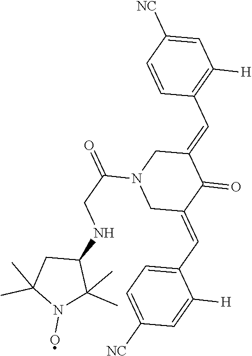

indicates a single enantiomer with the absolute configuration depicted.

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. When a single enantiomer is named or depicted by structure, the depicted or named enantiomer is present in an ee of at least or about: 50%, 60%, 70%, 80%, 90%, 95%, 96%, 97%, 98%, 99% or 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D4 . . . ) represent the respective fractions of each diastereomer in a mixture, such that D1+(D2+D3+D4 . . . )=1. When a single diastereomer is named or depicted by structure, the depicted or named diastereomer is present in a de of at least or about: 50%, 60%, 70%, 80%, 90%, 95%, 96%, 97%, 98%, 99% or 99.9%.

An isomer (e.g., diastereomer, enantiomer) can, in some embodiments, be provided substantially free of corresponding isomer(s) (e.g., diastereomer(s))/enantiomer). When an isomer is “substantially free” of corresponding isomer(s), the ee or de of the depicted or named compound is at least about 50%, for example, at least or about: 50%, 60%, 70%, 75%, 80%, 90%, 95%, 96%, 97%, 98%, 99% or 99.9%.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

An “amine” refers to a compound containing a nitrogen bound to a carbon through a single bond. A “primary amine” refers to an —NH₂ group, a “secondary amine” refers to a —NHR group wherein R is in a non-limiting example an alkyl, aryl, or heterocyclic group, but is not H, and a “tertiary amine” refers to a —NR₂ group wherein R is in a non-limiting example an alkyl, aryl, or heterocyclic group, but is not H. In some embodiments, a “protected amine” is described, which is referring to a either a secondary or tertiary amine. An “amide” refers to a —C(═O) NR₂, wherein R can be any group, including H. In some non-limiting embodiments, R in amides are typically H, alkyl, aryl, or heterocyclic groups.

An “amino acid” refers to a compound containing both an amine group and a carboxylic acid group. “Carboxylic acid” refers to a —COOH group. Some non-limiting examples of amino acids include: alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

A “phosphate salt” refers to a PO₄³⁻ group that has a cation as a counter ion. It is possible to have the phosphate group bound in multiple sites of the four oxygen atoms. It is common to see a singular phosphate group as —OP(═O)O₂²⁻, but it is also possible to see a phosphate group be bound at multiple sites, including being bound to multiple phosphate groups. An example of this can be found in the compound Nicotinamide adenine dinucleotide (NAD), which has two phosphate groups bridging together two nucleotides. Some non-limiting cations include, Na⁺, K⁺, Ca²⁺, Mg²⁺, NH⁴⁺ and the like.

The term “aryl,” as used herein, refers to an unsaturated ring system having a specific number of ring atoms. For a ring system to be aryl, the ring must have a delocalized pi system with alternating single and double bonds in a coplanar structure that follows Huckel's rule of aromaticity such that the structure is planar and has full conjugation of (4n+2) pi electrons, where n is an integer. In a non-limiting example, an aryl group is benzene, toluene, naphthalene, furan, indole, pyridine, and the like.

The term “heterocyclyl,” or “heterocylic” as used herein, refers to a saturated or partially saturated, non-aromatic, monocyclic or polycyclic (e.g., bicyclic, tricyclic) ring system having the specified number of ring atoms, wherein at least one carbon atom in the ring system has been replaced with a heteroatom independently selected from oxygen, sulfur and nitrogen. Thus, “(C₃-C₇)heterocyclyl” means a heterocyclyl having from 3-7 ring atoms. “Heterocyclyl” includes monocyclic rings, fused rings, bridged rings and spirocyclic rings. In some embodiments, heterocyclyl is (C₃-C₇)heterocyclyl, (C₅-C₆) heterocyclyl, (C₅)heterocyclyl or (C₆)heterocyclyl. In some embodiments, heterocyclyl (e.g., (C₃-C₇)heterocyclyl) is a saturated heterocyclyl.

Heterocyclyl can contain 1 to 7, 1 to 5, 1 to 3, 1 to 2, 1 or 2 heteroatoms. A heteroatom is a non-carbon atom such as nitrogen, oxygen, sulfur, phosphorous, and the like. A heterocyclyl can be attached at a heteroatom or a carbon atom, as valencies permit. Examples of heterocyclyl include, but are not limited to, azetidinyl, oxetanyl, piperidinyl, piperazinyl, pyrrolyl, pyrrolidinyl, imidazolidinyl, imidazolinyl, morpholinyl, tetrahydrofuranyl, tetrahydrothiophenyl, tetrahydrothiopyranyl, tetrahydropyranyl, 1,4-dioxanyl, 1,4-oxathianyl, hexahydropyrimidinyl, 3-azabicyclo[3.1.0]hexanyl, azepanyl, 3-azabicyclo[3.2.2]nonanyl, decahydroisoquinolinyl, 2-azaspiro[3.3]heptanyl, 2-oxa-6-azaspiro[3.3]heptanyl, 2,6-diazaspiro[3.3]heptanyl, 8-aza-bicyclo[3.2.1]octanyl, 3,8-diazabicyclo[3.2.1]octanyl, 3-oxa-8-aza-bicyclo[3.2.1]octanyl, 8-oxa-3-aza-bicyclo[3.2.1]octanyl, 2-oxa-5-aza-bicyclo[2.2.1]heptanyl, 2,5-diaza-bicyclo[2.2.1]heptanyl, 1,4-dioxa-8-aza-spiro[4.5]decanyl, 3-oxa-1,8-diazaspiro[4.5]decanyl, octahydropyrrolo[3,2-b]pyrrolyl, and the like.

A “carbocyclic” ring refers to a ring system comprising only carbon to carbon bonds. The bonds may be single, double, or triple bonds, but the ring system does not contain any heteroatoms within the ring. Carbocyclic rings may be alky or aryl. In some non-limiting examples, carbocyclic rings include benzene, cyclopentane, cyclohexane, and the like.

A “5- or 6-membered ring system” refers to a ring system of five or six atoms bound together as a ring. Some non-limiting examples of this include benzene, cyclopentane, furan, pyridine, and the like.

In one aspect, disclosed herein are 1-pyrrolidinyl-(PROXYL), and 1-piperidinyl-(TEMPO) ester derivatives of the general formula I and II as defined above, comprising one to two nitric oxide donor groups and a ROS degradation catalyst, i.e., a superoxide anion (O₂⁻) scavenger, for use in the treatment of preeclampsia and Covid-19 associated lung disease.

The compounds for use according to the present disclosure are prodrugs of the corresponding PROXYL and TEMPO compounds upon hydrolysis of the ester bond and those hydroxylamine compounds are then oxidized, in vivo, to their corresponding nitroxide derivatives, more particularly the PROXYL and TEMPO compounds of Formula I and II.

R1, R2 and R3 are independently an alkyl, aryl or heterocyclic groups. The term “alkyl” as used herein typically means a straight or branched saturated hydrocarbon radical having 1-10 carbon atoms and includes, e.g., methyl, ethyl, n-propyl, isopropyl, n-butyl, sec-butyl, isobutyl, tert-butyl, n-pentyl, isoamyl, 2,2-dimethylpropyl, n-hexyl, n-heptyl, n-octyl, n-nonyl, n-decyl, more preferably (C₁-C₄) alkyl groups, most preferably methyl, ethyl, propyl and butyl.

TABLE 1
Structures I, II and III indicating PROXYL,
TEMPO and piperidone derivatives

| | |
|---|---|
| 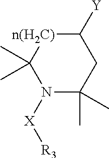 | I |
| 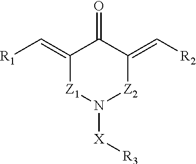 | II |
| 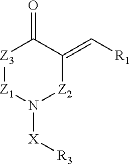 | III |

In certain particular embodiments, the compound for use according to the present disclosure is a compound of the formula I in Table 1, wherein either X is selected from O^{·}, OH, CO, OCO, CO(CH2)n, OCO(CH2)n—, CO(CH2)nCO, OCO(CH2)nCO—, wherein R3 is substituted amines such as methylamine, dimethyl, trimethyl, ethylamine, diethyl, isopropylamine, pyrrolidine, piperazine, morpholine and nitroxide containing amines such as 3-amino-proxyl, 3-aminomethyleneproxyl, 4-amino-TEMPO, and amino acids such as glycine, valine, arginine, proline and cystine linked via amides and phosphate salts or alkyl esters carrying substituted amines, amino acids, amides and phosphate salts;
Y is selected from H, CO, —OH, CH2OH, —CO2H, —ONO₂, —CH₂ONO₂, —CH₂CH₂ONO₂, CH═CH—CH₂ONO₂, COOCH₂CH₂ONO₂ and —COOalkyl where said alkyl may be optionally substituted, for example, with —ONO2.

Z1, Z2, and Z3 are independently selected from CH2, —CF2, CMe2, CO, CHCONHR3, CH—COCH═CHR1, CH—COCH═CHR2, C═CH—R1 or C═CH—R2 or Z1 and Z2 taken together form a 5 or 6 membered carbocyclic or heterocyclic ring or

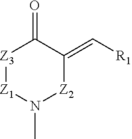

ring is selected from the following rings:

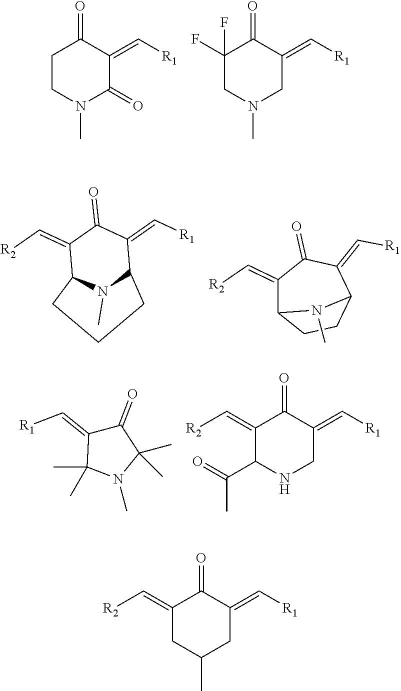

n is 1 to 4.

R1 and R2 are independently an alkyl, a 5 or 6 membered aromatic heterocyclic or carbocyclic rings optionally substituted with OH, OMe, F, CF3, CN, NO2, CO2Et or SO2Me or other electron withdrawing groups.

In certain particular embodiments, the compound for use according to the present disclosure is a compound of the formula II in Table 1, wherein Y is selected from H, CO, —OH, CH2OH, —CO2H, —ONO₂, —CH₂ONO₂, —CH₂CH₂ONO₂, CH═CH—CH₂ONO₂, COOCH₂CH₂ONO₂ and —COOalkyl where said alkyl may be optionally substituted, for example, with —ONO2.

TABLE 2
Compounds of the general formula I herein identified 1a-h and 2a-p,
and general formula of II and III identified 3a-i to 20a-i for use
according to the present disclosure.

| | |
|---|---|
| 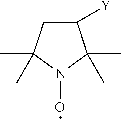 | la-b |
| 1a: Y = CH2OH | |
| 1b: Y = CH2ONO2 | |
| 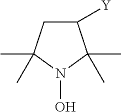 | 1c-d |
| 1c: Y = CH2OH | |
| 1d: Y = CH2ONO2 | |
| 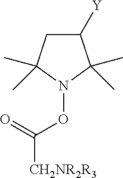 | 1e-f |
| 1e: Y = CH2OH; | |
| NR2R3 = —NMe2 | |
| 1f: Y = CH2ONO2; | |
| NR2R3 = NMe2 | |
| 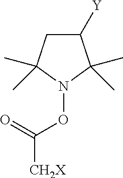 | 1g-h |
| 1g: Y = CH2OH, | |
| X = CH2CONH-Arginine(CO2Et) | |
| 1h: Y = CH2ONO2, | |
| X = CH2CONH-Arginine (CO2Et) | |
| 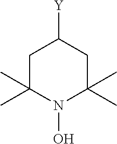 | 2a-d |
| 2a: Y = OH | |
| 2b: Y = ONO2 | |
| 2c: Y = CH2OH | |
| 2d: Y = CH2ONO2 | |
| 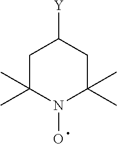 | 2e-h |
| 2e: Y = OH | |
| 2f: Y = ONO2 | |
| 2g: Y = CH2OH | |
| 2h: Y = CH2ONO2 | |
| 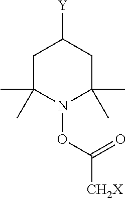 | 2i-l |
| 2i: Y = OH, X = NMe2 | |
| 2j: Y = ONO2, X = NMe2 | |
| 2k: Y = CH2OH, X = N + Me3 | |
| 2l: Y = CH2ONO2, X = N + Me3 | |
| 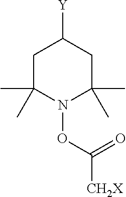 | 2m-p |
| 2m: Y = OH; | |
| X = CH2CONH(ARG)CO2Et | |
| 2n: Y = ONO2; | |
| X = CH2CONH(ARG)CO2Et | |
| 2o: Y = CH2OH; | |
| X = CH2CONH-valine(CO2Et) | |
| 2p: Y = CH2ONO2; | |
| X = CH2CONH-valine (CO2Et) | |
| 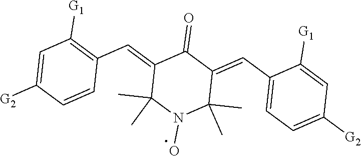 | 3a-i |
| 3a: G1 = H, G2 = F | |
| 3b: G1 = CN, G2 = F | |
| 3c: G1 = F, G2 = F | |
| 3d: G1 = H, G2 = CN | |
| 3e: G1 = F, G2 = CN | |
| 3f: G1 = CN, G2 = CN | |
| 3g: G1 = H, G2 = CF3 | |
| 3h: G1 = H, G2 = CO2Et | |
| 3i: G1 = H, G2 = NO2 | |
| 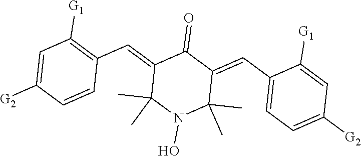 | 4a-i |
| 4a: G1 = H, G2 = F | |
| 4b: G1 = CN, G2 = F | |
| 4c: G1 = F, G2 = F | |
| 4d: G1 = H, G2 = CN | |
| 4e: G1 = F, G2 = CN | |
| 4f: G1 = CN, G2 = CN | |
| 4g: G1 = H, G2 = CF3 | |
| 4h: G1 = H, G2 = CO2Et | |
| 4i: G1 = H, G2 = NO2 | |
| 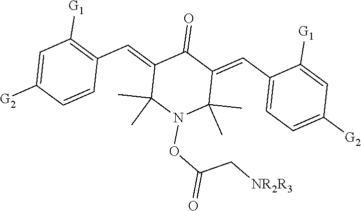 | 5a-i |
| 5a: G1 = H, G2 = F | |
| 5b: G1 = CN, G2 = F | |
| 5c: G1 = F, G2 = F | |
| 5d: G1 = H, G2 = CN | |
| 5e: G1 = F, G2 = CN | |
| 5f: G1 = CN, G2 = CN | |
| 5g: G1 = H, G2 = CF3 | |
| 5h: G1 = H, G2 = CO2Et | |
| 5i: G1 = H, G2 = NO2 | |
| 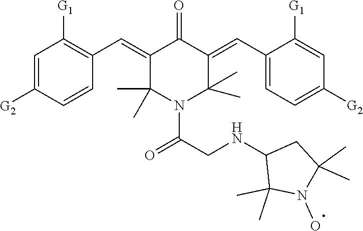 | 6a-i |
| 6a: G1 = H, G2 = F | |
| 6b: G1 = CN, G2 = F | |
| 6c: G1 = F, G2 = F | |
| 6d: G1 = H, G2 = CN | |
| 6e: G1 = F, G2 = CN | |
| 6f: G1 = CN, G2 = CN | |
| 6g: G1 = H, G2 = CF3 | |
| 6h: G1 = H, G2 = CO2Et | |
| 6i: G1 = H, G2 = NO2 | |
| 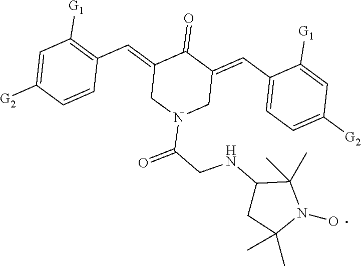 | 7a-i |
| 7a: G1 = H, G2 = F | |
| 7b: G1 = CN, G2 = F | |
| 7c: G1 = F, G2 = F | |
| 7d: G1 = H, G2 = CN | |
| 7e: G1 = F, G2 = CN | |
| 7f: G1 = CN, G2 = CN | |
| 7g: G1 = H, G2 = CF3 | |
| 7h: G1 = H, G2 = CO2Et | |
| 7i: G1 = H, G2 = NO2 | |
| 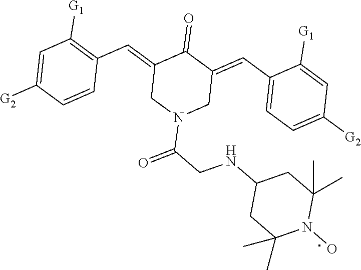 | 8a-i |
| 8a: G1 = H, G2 = F | |
| 8b: G1 = CN, G2 = F | |
| 8c: G1 = F, G2 = F | |
| 8d: G1 = H, G2 = CN | |
| 8e: G1 = F, G2 = CN | |
| 8f: G1 = CN, G2 = CN | |
| 8g: G1 = H, G2 = CF3 | |
| 8h: G1 = H, G2 = CO2Et | |
| 8i: G1 = H, G2 = NO2 | |
| 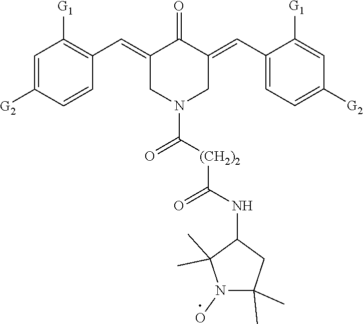 | 9a-i |
| 9a: G1 = H, G2 = F | |
| 9b: G1 = CN, G2 = F | |
| 9c: G1 = F, G2 = F | |
| 9d: G1 = H, G2 = CN | |
| 9e: G1 = F, G2 = CN | |
| 9f: G1 = CN, G2 = CN | |
| 9g: G1 = H, G2 = CF3 | |
| 9h: G1 = H, G2 = CO2Et | |
| 9i: G1 = H, G2 = NO2 | |
| 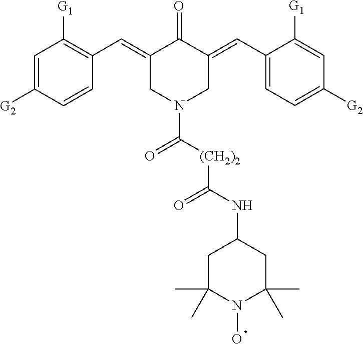 | 10a-i |
| 10a: G1 = H, G2 = F | |
| 10b: G1 = CN, G2 = F | |
| 10c: G1 = F, G2 = F | |
| 10d: G1 = H, G2 = CN | |
| 10e: G1 = F, G2 = CN | |
| 10f: G1 = CN, G2 = CN | |
| 10g: G1 = H, G2 = CF3 | |
| 10h: G1 = H, G2 = CO2Et | |
| 10i: G1 = H, G2 = NO2 | |
| 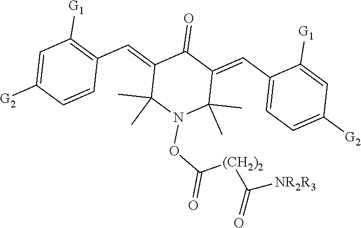 | 11a-i |
| 11a: G1 = H, G2 = F | |
| 11b: G1 = CN, G2 = F | |
| 11c: G1 = F, G2 = F | |
| 11d: G1 = H, G2 = CN | |
| 11e: G1 = F, G2 = CN | |
| 11f: G1 = CN, G2 = CN | |
| 11g: G1 = H, G2 = CF3 | |
| 11h: G1 = H, G2 = CO2Et | |
| 11i: G1 = H, G2 = NO2 | |
| 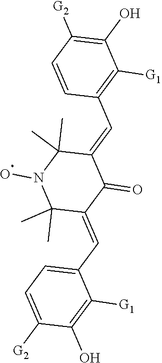 | 12 |
| 12: G1 = H, G2 = OMe | |
| 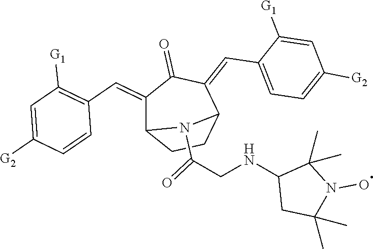 | 13a-i |
| 13a: G1 = H, G2 = F | |
| 13b: G1 = CN, G2 = F | |
| 13c: G1 = F, G2 = F | |
| 13d: G1 = H, G2 = CN | |
| 13e: G1 = F, G2 = CN | |
| 13f: G1 = CN, G2 = CN | |
| 13g: G1 = H, G2 = CF3 | |
| 13h: G1 = H, G2 = CO2Et | |
| 13i: G = H, G2 = NO2 | |
| 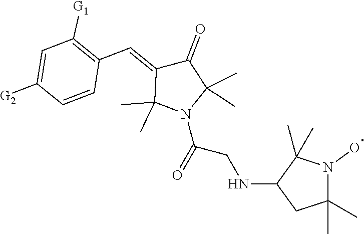 | 14a-i |
| 14a: G1 = H, G2 = F | |
| 14b: G1 = CN, G2 = F | |
| 14c: G1 = F, G2 = F | |
| 14d: G1 = H, G2 = CN | |
| 14e: G1 = F, G2 = CN | |
| 14f: G1 = CN, G2 = CN | |
| 14g: G1 = H, G2 = CF3 | |
| 14h: G1 = H, G2 = CO2Et | |
| 14i: G1 = H, G2 = NO2 | |
| 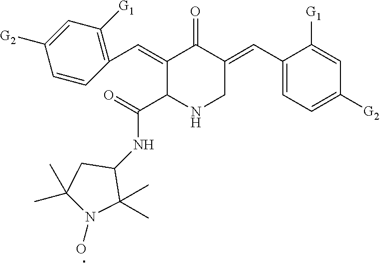 | 15a-i |
| 15a: G1 = H, G2 = F | |
| 15b: G1 = CN, G2 = F | |
| 15c: G1 = F, G2 = F | |
| 15d: G1 = H, G2 = CN | |
| 15e: G1 = F, G2 = CN | |
| 15f: G1 = CN, G2 = CN | |
| 15g: G1 = H, G2 = CF3 | |
| 15h: G1 = H, G2 = CO2Et | |
| 15i: G1 = H, G2 = NO2 | |
| 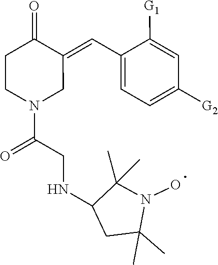 | 16a-i |
| 16a: G1 = H, G2 = F | |
| 16b: G1 = CN, G2 = F | |
| 16c: G1 = F, G2 = F | |
| 16d: G1 = H, G2 = CN | |
| 16e: G1 = F, G2 = CN | |
| 16f: G1 = CN, G2 = CN | |
| 16g: G1 = H, G2 = CF3 | |
| 16h: G1 = H, G2 = CO2Et | |
| 16i: G1 = H, G2 = NO2 | |
| 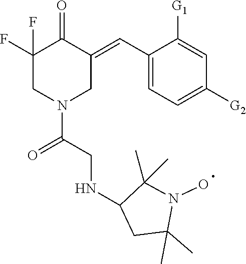 | 17a-i |
| 17a: G1 = H, G2 = F | |
| 17b: G1 = CN, G2 = F | |
| 17c: G1 = F, G2 = F | |
| 17d: G1 = H, G2 = CN | |
| 17e: G1 = F, G2 = CN | |
| 17f: G1 = CN, G2 = CN | |
| 17g: G1 = H, G2 = CF3 | |
| 17h: G1 = H, G2 = CO2Et | |
| 17i: G1 = H, G2 = NO2 | |
| 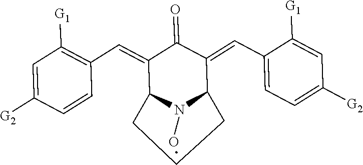 | 18a-i |
| 18a: G1 = H, G2 = F | |
| 18b: G1 = CN, G2 = F | |
| 18c: G1 = F, G2 = F | |
| 18d: G1 = H, G2 = CN | |
| 18e: G1 = F, G2 = CN | |
| 18f: G1 = CN, G2 = CN | |
| 18g: G1 = H, G2 = CF3 | |
| 18h: G1 = H, G2 = CO2Et | |
| 18i: G1 = H, G2 = NO2 | |
| 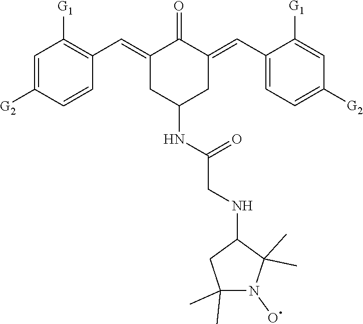 | 19a-i |
| 19a: G1 = H, G2 = F | |
| 19b: G1 = CN, G2 = F | |
| 19c: G1 = F, G2 = F | |
| 19d: G1 = H, G2 = CN | |
| 19e: G1 = F, G2 = CN | |
| 19f: G1 = CN, G2 = CN | |
| 19g: G1 = H, G2 = CF3 | |
| 19h: G1 = H, G2 = CO2Et | |
| 19i: G1 = H, G2 = NO2 | |
| 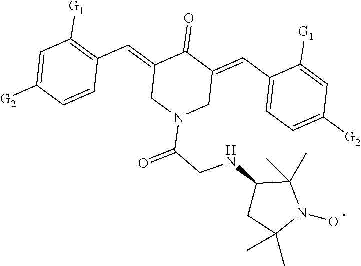 | 20a-i |
| 20a: G1 = H, G2 = F | |
| 20b: G1 = CN, G2 = F | |
| 20c: G1 = F, G2 = F | |
| 20d: G1 = H, G2 = CN | |
| 20e: G1 = F, G2 = CN | |
| 20f: G1 = CN, G2 = CN | |
| 20g: G1 = H, G2 = CF3 | |
| 20h: G1 = H, G2 = CO2Et | |
| 20i: G1 = H, G2 = NO2 | |
| 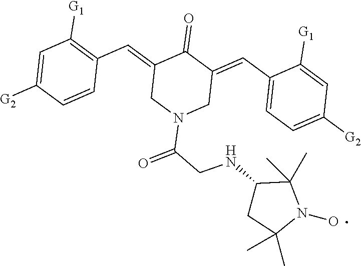 | 21a-b |
| 21a: G1 = H, G2 = F | |
| 21b: G1 = CN, G2 = F | |
| 21c: G1 = F, G2 = F | |
| 21d: G1 = H, G2 = CN | |
| 21e: G1 = F, G2 = CN | |
| 21f: G1 = CN, G2 = CN | |
| 21g: G1 = H, G2 = CF3 | |
| 21h: G1 = H, G2 = CO2Et | |
| 21i: G1 = H, G2 = NO2 | |

*Similar compounds in which each one of the amines (—NR2R3) are primary, secondary, and tertiary amines, 3-amino-proxyl, 4-amino-TEMPO or amino acids.

The compounds for use according to the present disclosure may be synthesized according to any technology or procedure known in the art, or as described in experimental section.

The compounds of the general formula I may have one or more asymmetric centers, and may accordingly exist both as enantiomers, i.e., optical isomers (R, S, or racemate, wherein a certain enantiomer may have an optical purity of 90%, 95%, 99% or more) and as diastereoisomers. Specifically, those chiral centers may be, e.g., in each one of the carbon atoms of the 1-pyrrolidinyl ester derivative, of the general formulas I. It should be understood that the present disclosure encompasses the use of all such enantiomers, isomers and mixtures thereof, as well as pharmaceutically acceptable salts thereof.

Optically active forms of the compounds of the general formula I may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

In another aspect, the present disclosure provides a pharmaceutical composition comprising a compound of the general formula I as defined in any one of the embodiments above, or an enantiomer, diastereomer, racemate, or a pharmaceutically acceptable salt thereof (herein also referred to as the “active agent”), and a pharmaceutically acceptable carrier, for treatment of Covid-19 associated pneumonia. Particular such pharmaceutical compositions comprise, as an active agent, a compound selected from the compounds of Table 2 above, e.g., compounds 1 to 12, or an enantiomer, diastereomer, racemate, or a pharmaceutically acceptable salt thereof.

The pharmaceutical compositions of the present disclosure can be provided in a variety of formulations, e.g., in a pharmaceutically acceptable form and/or in a salt form, as well as in a variety of dosages.

In one embodiment, the pharmaceutical composition of the present disclosure comprises a non-toxic pharmaceutically acceptable salt of a compound of the general formula I. Suitable pharmaceutically acceptable salts include acid addition salts such as, without being limited to, the mesylate salt, the maleate salt, the fumarate salt, the tartrate salt, the hydrochloride salt, the hydrobromide salt, the mesylate salt, the p-toluenesulfonate salt, the benzenesulfonate salt, the benzoate salt, the acetate salt, the phosphate salt, the sulfate salt, the citrate salt, the carbonate salt, and the succinate salt. Additional pharmaceutically acceptable salts include salts of ammonium (NH₄⁺) or an organic cation derived from an amine of the formula R₄N⁺, wherein each one of the Rs independently is selected from H, C₁-C₁₀, preferably C₁-C₆ alkyl, such as methyl, ethyl, propyl, isopropyl, n-butyl, sec-butyl, isobutyl, tert-butyl, n-pentyl, 2,2-dimethylpropyl, and n-hexyl Furthermore, where the compounds of the general formula I carry an acidic moiety, suitable pharmaceutically acceptable salts thereof may include metal salts such as alkali metal salts, e.g., lithium, sodium or potassium salts, and alkaline earth metal salts, e.g., calcium or magnesium salts.

Pharmaceutically acceptable salts of the compound for use according to the present disclosure may be formed by conventional means, e.g., by reacting a free base form of the active agent, i.e., the compound of the general formula I, with one or more equivalents of the appropriate acid in a solvent or medium in which the salt is insoluble, or in a solvent such as water which is removed in vacuo or by freeze drying, or by exchanging the anion/cation of an existing salt for another anion/cation on a suitable ion exchange resin.

The pharmaceutical compositions disclosed herein can be formulated for any suitable route of administration, but they are preferably formulated for parenteral, e.g., intravenous, intraarterial, intramuscular, intraperitoneal, intrathecal, intrapleural, intratracheal, or subcutaneous administration. In certain embodiments, the compositions are formulated for intramuscular injections and are thus suitable, inter alia, for emergent use. The dosage will depend on the state of the patient and will be determined as deemed appropriate by the practitioner.

The pharmaceutical composition of the present disclosure may be in the form of a sterile injectable aqueous or oleaginous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), Tween-80, and isotonic sodium chloride solution.

Pharmaceutical compositions according to the present disclosure, when formulated for administration route other than parenteral administration, may be in a form suitable for oral use, e.g., as tablets, troches, lozenges, aqueous, or oily suspensions, dispersible powders or granules, emulsions, hard or soft capsules, or syrups or elixirs.

Pharmaceutical compositions intended for oral administration should be formulated so as to inhibit the release of the active agent in the stomach, i.e., delay the release of the active agent until at least a portion of the dosage form has traversed the stomach, in order to avoid the acidity of the gastric contents from hydrolyzing the active agent to its highly water insoluble form, i.e., its corresponding 1-pyrrolidinyloxy, 1-piperidinyloxy or 1-azepanyloxy derivative. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly(methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

Pharmaceutical compositions intended for oral administration may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the present disclosure may also be in the form of oil-in-water emulsion.

Oral pharmaceutical compositions according to the present disclosure may be formulated for controlled release of the active agent. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

Another contemplated formulation is depot systems, based on biodegradable polymers, wherein as the polymer degrades, the active ingredient is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

In yet another aspect, the present disclosure relates to use of a compound of the general formula I as defined in any one of the embodiments above, or an enantiomer, diastereomer, racemate, or a pharmaceutically acceptable salt thereof, for the preparation of a pharmaceutical composition for treatment of Covid-19 associated pneumonia. In certain embodiments, the compound used for the preparation of a pharmaceutical composition as disclosed herein is selected from the compounds of Tables 1-2 above, e.g., compound 1-12, or an enantiomer, diastereomer, racemate, or a pharmaceutically acceptable salt thereof.

In a further aspect, the present disclosure relates to a method for treatment of Covid-19 associated pneumonia in an individual in need thereof, comprising administering to said individual an effective amount of a compound of the general formula I as defined in any one of the embodiments above, or an enantiomer, diastereomer, racemate, or a pharmaceutically acceptable salt thereof. In certain embodiments, the compound administered according to the method disclosed herein is selected from the compounds of Tables 1-2 above, e.g., compound 1-12, or an enantiomer, diastereomer, racemate, or a pharmaceutically acceptable salt thereof.

In a further aspect, the present disclosure relates to a method for treatment or prevention of preeclampsia.

In a further aspect, the present disclosure relates to a method for treatment of pulmonary hypertension, including but not limited to primary pulmonary hypertension, pulmonary arterial hypertension (PAH), pulmonary hypertension caused by lung injury, heart disease or other health conditions or prevention of cancer.

In a further aspect, cancer refers to multiple myeloma, leukemia, alveolar rhabdomyosarcoma, melanoma, lymphoma, astrocytoma, biphasic synovial sarcoma, bladder carcinoma, bone cancer, breast cancer, cecum adenocarcinoma, cervical cancer, CNS cancer, colon cancer, colorectal cancer, duodenal adenocarcinoma, embryonal rhabdomyosarcoma, endometrial cancer, epithelioid sarcoma, fibrosarcoma, gastric cancer, signet ring cell gastric adenocarcinoma, gestational choriocarcinoma, glioblastoma, hereditary thyroid gland medullary carcinoma, hypopharyngeal squamous cell carcinoma, invasive ductal carcinoma, liposarcoma, lung cancer, neuroblastoma, osteosarcoma, ovarian cancer, uterine cancer, pancreatic cancer, papillary renal cell carcinoma, prostate cancer, rectal adenocarcinoma, medulloblastoma, renal cancer, testicular embryonal carcinoma, and tongue squamous cell carcinoma.

The present disclosure will now be illustrated by the following non-limiting Examples.

### Example 1: Synthesis of Compounds 1-12

Compounds 1-12 may be synthesized as shown in Scheme 1 to 3 starting from proxyl and TEMPOL substituted with alcohol, acid and amino groups (Ref. 4 to 8).

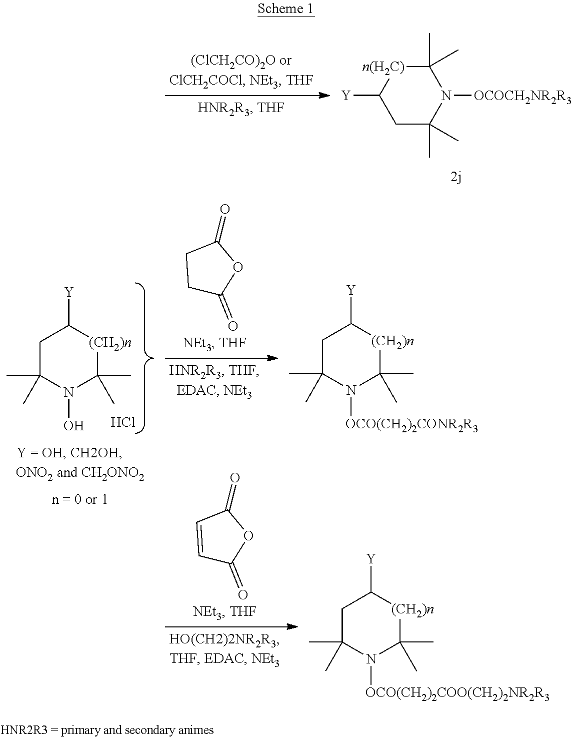

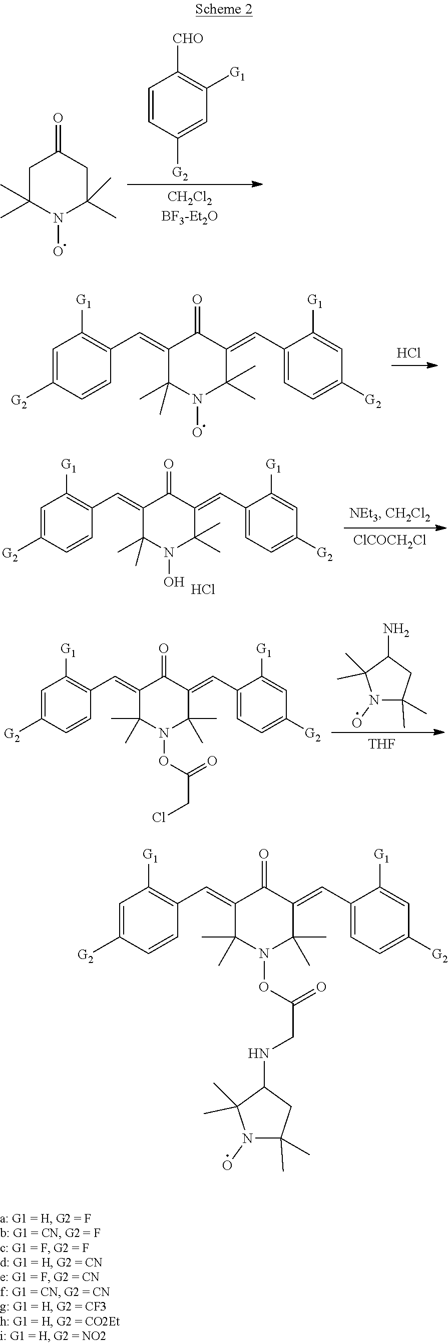

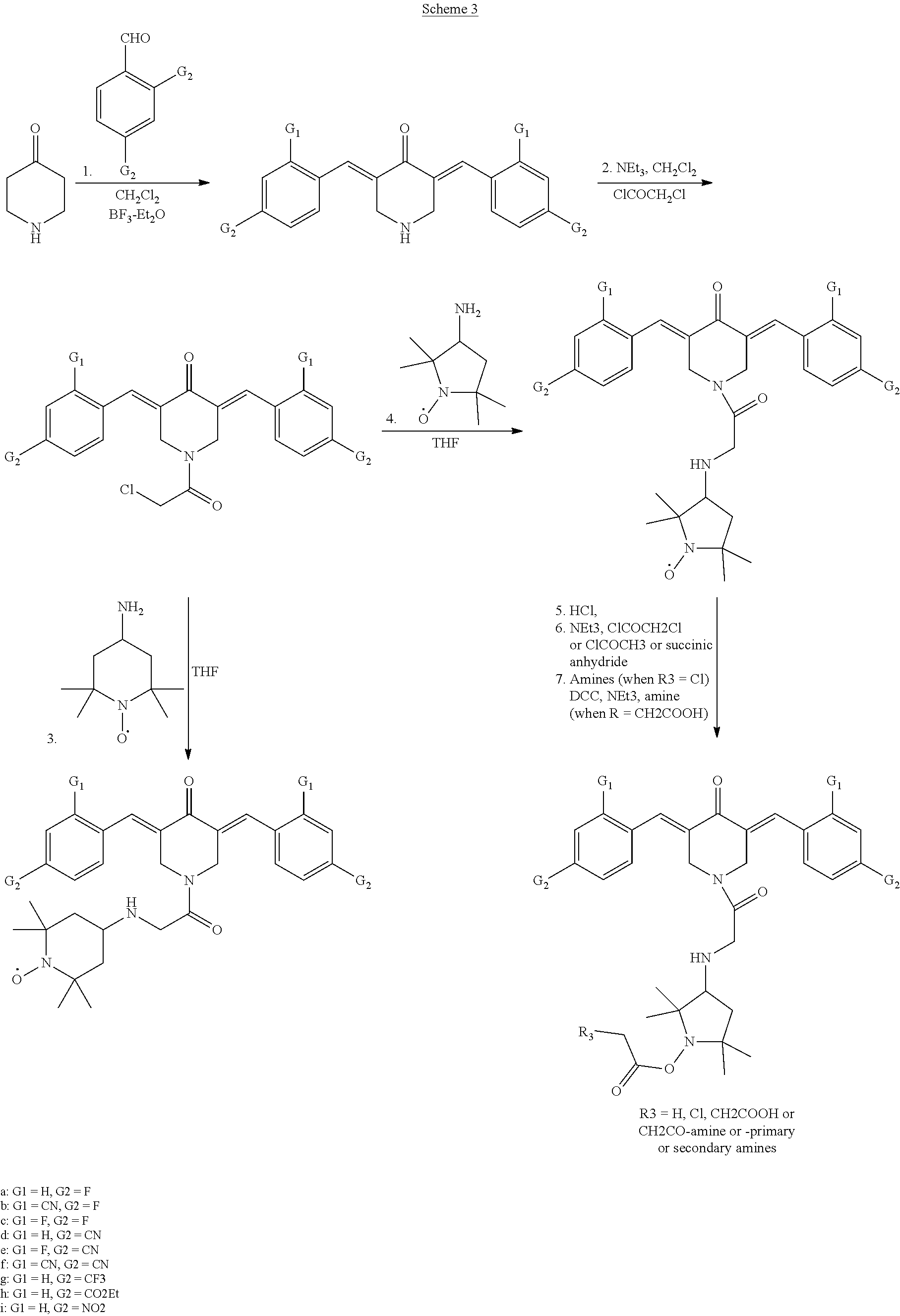

### Example 2: Cell Viability by MTT Assay

Cell viability was determined by a colorimetric assay using 3-(4,5-dimethylthiazol-2-yl)-2,5-diphenyltetrazolium bromide (MTT). In the mitochondria of living cells, yellow MTT undergoes a reductive conversion to formazan, producing a purple color. Cells, grown to ˜80% confluence in 75-mm flasks, were trypsinized, counted, seeded in 96-well plates with an average population of 7,000 cells per well, incubated overnight, and then treated with Compound 6b or HO-3867 [Ref 3] for 24 hours. HO-3867 is cytotoxic to other ovarian cancer cell lines. The cytotoxic effects of AKT-100 (7d) were evaluated and compared with that of HO-3867 on the viability of ovarian cancer cells (PEO1, ECC1 and KLE with mutant p53 and BRCA) [Ref 9]. Compound 7d exhibited significantly higher toxicity when compared with HO-3867 (Ref 10) as shown in Table 3.

TABLE 3

| Cells | PE01 | ECC-1 | KLE |
|---|---|---|---|
| Compound 7d | 200 nM | 300 nM | 1500 nM |
| HO-3867 | 1700 nM | 1500 nM | 4000 nM |

### REFERENCES

1. (a) Zsengellér Z K, Lo A, Tavasoli M, Pernicone E, Karumanchi S A, Rosen S. Soluble fms-Like Tyrosine Kinase 1 Localization in Renal Biopsies of CKD. Kidney Int Rep. 2019;4 (12): 1735-41. (b) Zsengeller Z K, Rajakumar A, Hunter J T, Salahuddin S, Rana S, Stillman I E, et al. Trophoblast mitochondrial function is impaired in preeclampsia and correlates negatively with the expression of soluble fms-like tyrosine kinase 1. Pregnancy Hypertens. 2016;6 (4): 313-9.
2. Marcin Lewandowski and Krzysztof Gwozdzinski, Nitroxides as Antioxidants and Anticancer Drugs. Int. J. Mol. Sci. 2017, 18, 2490; doi: 10.3390/ijms18112490.
3. (a) Kelly Rath et al, HO-3867, a Safe STAT3 Inhibitor, Is Selectively Cytotoxic to Ovarian Cancer, Cancer Res. 2014 Apr. 15; 74 (8): 2316-2327. doi: 10.1158/0008-5472. CAN-13-2433. (b) Periannan Kuppusamy, Compositions and methods for inhibition of cancers. US 2012/0316203 A1.
4. Prakash Jagtap et al., Multifunctional nitroxide derivatives and uses thereof. U.S. Pat. No. 9,522,903, 2016.
5. Salzman et al., Substituted 1-pyrrolidinyloxy, 1-piperidinyloxy and 1-azepanyloxy compounds for treating diseases associated with oxidative stress or endothelial dysfunction, 9604932, 2014.
6. Kalid O. et al, PTX80, a novel compound targeting the autophagy receptor p62/SQSTM1 for treatment of cancer, Chem Biol Drug Des. 2022, 100(5):623-638.
7. Kalid O. et al. Compounds for inhibiting protein degradation and method of use thereof in the treatment of cancer. WO 2019/171379 A1
8. Selvendiran et al, Anticancer efficacy of a difluorodiarylidenyl piperidone (HO-3867) in human ovarian cancer cells and tumor xenografts, Mol Cancer Ther. 2010, 9(5):1169-79.
9. Davor E. et al, The Synthetic Curcumin Analog HO-3867 Rescues Suppression of PLAC1 Expression in Ovarian Cancer Cells, Pharmaceuticals 2021, 14, 942. https://doi.org/10.3390/ph14090942.
10. Bazzaro M. et al. Dienone Compounds: Targets and Pharmacological Responses, J. Med. Chem. 2020, 63, 15075-15093.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method for treating preeclampsia or preeclampsia associated conditions comprising eclampsia, haemorrhagic stroke, haemolysis, elevated liver enzymes and low platelet count (HELLP) syndrome, placental abruption, renal failure, or pulmonary oedema, the method comprising administering to a patient in need, a compound of Formula I: 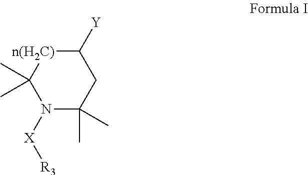
or enantiomers, diastereomers, racemates, or a pharmaceutically acceptable salt thereof, wherein:
X is selected from O· or —OH or H wherein R3 is absent or X is selected from CO;
R3 is substituted H or C1 to C10 alkyl or aryl or heterocyclic rings;
Y is selected from —OH, CH2OH, —ONO2, and —CH2ONO2, where said alkyl may be optionally substituted, for example, with —ONO2;
n is 0 to 4.

2. A compound of Formula 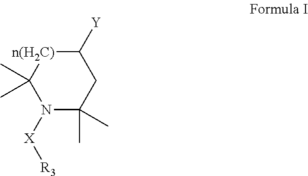
or enantiomers, diastereomers, racemates, or a pharmaceutically acceptable salt thereof,
wherein:
X is selected from O· or OH or H wherein R3 is absent or X is selected from CO, OCO, SO2, OSO2, CONH—, OCONH—, —CO-aryl or heterocyclic ring, —OCO-aryl or heterocyclic ring, CO(CH2)n, CO(CH2)nCO, OCO(CH2)nCO, OCO(CH2)n—;
R3 is substituted amines such as methylamine, dimethyl, diethyl, trimethyl, ethylamine, propylamine, isopropylamine, pyrrolidine, piperazine, morpholine, and nitroxide containing amines such as 3-amino-proxyl, 3-aminomethyleneproxyl, 4-amino-TEMPO, and amino acids such as glycine, valine, arginine, proline and cystine linked via amides and phosphate salts or alkyl esters carrying substituted amines, amino acids, amides and phosphate salts;
Y is selected from H, —O, —OH, CH2OH, —CO2H, —ONO2, —CH2ONO2, —CH2CH2ONO2, CH═CH—CH2ONO2, COOCH2CH2ONO2 and —COOalkyl where said alkyl may be optionally substituted, for example, with —ONO2;
n is 0 to 4.

3. A compound of Formula II or III: 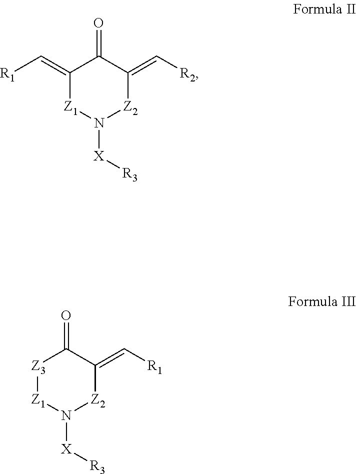
or enantiomers, diastereomers, racemates, or a pharmaceutically acceptable salt thereof,
wherein:
X is selected from O·, CO, OCO, SO2, OSO2, COO, OCOO, CONH—, OCONH, OH,
—CO-aryl or heterocyclic ring, OCO-aryl or heterocyclic, CO(CH2)n, CO(CH2)nCO, OCO(CH2)nCO, OCO(CH2)n—, wherein R3 is absent when X is O· or OH or H;
R3 is substituted amines such as methylamine, dimethyl, trimethyl, ethylamine, diethyl, propylamine, isopropylamine, pyrrolidine, piperazine, morpholine, and nitroxide containing amines such as 3-amino-proxyl, 3-aminomethyleneproxyl, 4-amino-TEMPO, and amino acids such as glycine, valine, arginine, proline and cystine linked via amides and phosphate salts or alkyl esters carrying substituted amines, amino acids, amides and phosphate salts;
Z1, Z2, and Z3 are independently selected from CH2, —CF2, CMe2, CO, CHCONH—R3, CH—COCH═CHR1, CH—COCH═CHR2, C═CH—R1 or C—CH-R2 or Z1 and Z2 taken together form a 5 or 6 membered carbocyclic or heterocyclic ring or 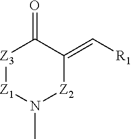
ring is selected from the following rings: 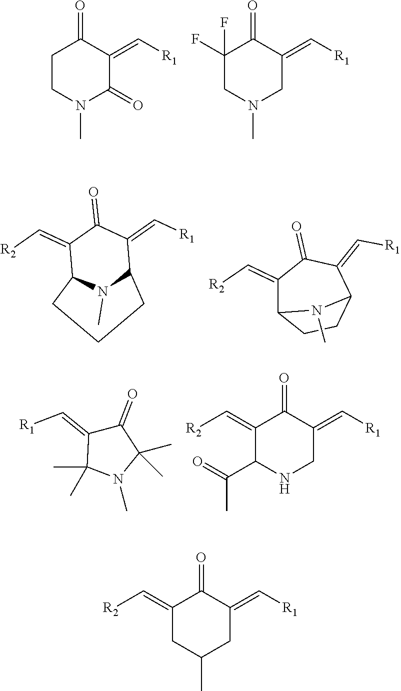
n is 0 to 4; and
R1 and R2 are either on same side of the double bond, or on opposite sides of the double bond, with E/E, E/Z or Z/Z configuration and any alkyl, 5 or 6 membered aromatic heterocyclic and carbocyclic rings substituted with H, OH, OMe, F, CF3, CN, NO₂, CO₂Et or SO₂Me groups.

4. The compound of claim 2, wherein the compound is formulated into a pharmaceutical composition comprising a pharmaceutically acceptable excipient.

5. The compound of claim 3, wherein the compound is formulated into a pharmaceutical composition and further comprises at least one chemotherapeutic.

6. The compound of claim 5, wherein the at least one chemotherapeutic comprises one or more of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride, Olaparib, veliparib, rucaparib, talazoparib, or niraparib.

7. A method for treating preeclampsia or preeclampsia associated conditions comprising eclampsia, haemorrhagic stroke, haemolysis, elevated liver enzymes and low platelet count (HELLP) syndrome, placental abruption, renal failure, or pulmonary oedema, the method comprising administering the compound of claim 2 to a patient in need.

8. The method of claim 7, wherein the compound is administered as a pharmaceutical composition having a pharmaceutically acceptable excipient.

9. A method for preventing cancer, the method comprising administering the compound of claim 3.

10. The method of claim 9, wherein the cancer is from a cancer selected from multiple myeloma, leukemia, alveolar rhabdomyosarcoma, melanoma, lymphoma, astrocytoma, biphasic synovial sarcoma, bladder carcinoma, bone cancer, breast cancer, cecum adenocarcinoma, cervical cancer, CNS cancer, colon cancer, colorectal cancer, duodenal adenocarcinoma, embryonal rhabdomyosarcoma, endometrial cancer, epithelioid sarcoma, fibrosarcoma, gastric cancer, signet ring cell gastric adenocarcinoma, gestational choriocarcinoma, glioblastoma, hereditary thyroid gland medullary carcinoma, hypopharyngeal squamous cell carcinoma, invasive ductal carcinoma, liposarcoma, lung cancer, neuroblastoma, osteosarcoma, ovarian cancer, uterine cancer, pancreatic cancer, papillary renal cell carcinoma, prostate cancer, rectal adenocarcinoma, medulloblastoma, renal cancer, testicular embryonal carcinoma, and tongue squamous cell carcinoma.

11. The method of claim 9, further comprising at least one chemotherapeutic.

12. The method of claim 11, wherein the at least one chemotherapeutic comprises one or more of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride, Olaparib, veliparib, rucaparib, talazoparib, or niraparib.
